(19) United States
(12) Patent Application Publication
(10) Pub. No.: US 2007/0083156 A1
(43) Pub. Date: Apr. 12, 2007

(54) Subcutaneous needle connection system

(75) Inventors: Rudolph Muto, Lawrence, MA (US); Steve A. Herweck, Nashua, NH (US)

Correspondence Address: LAHIVE & COCKFIELD, LLP, ONE POST OFFICE SQUARE, BOSTON, MA, 02109-2127, US

(73) Assignee: ATRIUM MEDICAL CORPORATION, Hudson, NH (US)

(21) Appl. No.: 11/246,462

(22) Filed: Oct. 6, 2005

(51) Int. Cl. A61M 31/00

(52) U.S. Cl. 604/93.01

(57) ABSTRACT

An apparatus for establishing a re-usable, recurring, mechanical connection to an organ within a patient is provided. A subcutaneous needle connection system for providing bidirectional, straight and turbulent-free fluid flow access to a vascular system of a patient includes a housing defining a needle access opening for receiving a needle, a cannula opening in communication with the vascular system and a passageway connecting the needle access opening to the cannula opening. The housing can additionally have a second needle access opening connected to a second cannula opening via a second passageway. The subcutaneous needle connection system allows for blood to be removed from the vascular system via one cannula opening, passed to a hemodialysis system via a needle inserted in an associated needle access opening, treated using the hemodialysis system, and returned to the vascular system via the other needle access opening and associated cannula opening. The implantable needle connection system provides enhanced mechanical stability under the skin, improved fluid flow dynamics, improved needle hole healing potential of the skin, and improved needle edge interfacing to minimize flow turbulence and blood cell trauma during use.

### FIELD OF THE INVENTION

The present invention relates to a surgical implant to provide a subcutaneous connection to a vascular system of a patient. More particularly the present invention relates to a subcutaneous needle connection system for providing repeated access to the vascular system of a patient.

### BACKGROUND OF THE INVENTION

A number of patients today undergo recurring medical procedures requiring repeated skin penetration to access the patient's vascular system and internal organs, including organs contained within the mediastinal, chest, abdominal and peritoneal cavities. One such recurring medical procedure is hemodialysis, which is used to treat kidney failure by removing harmful wastes and excess salts and fluid from a patient's blood.

Currently, over one million patients worldwide suffer from End State Renal Disease (ESRD) conditions and require some form of daily or thrice weekly dialysis treatment via needle or catheter access. Peritoneal Dialysis is one form of dialysis treatment requiring needle or catheter access whereby fluids placed into the peritoneal cavity via a temporary or permanently placed access catheter provide osmotic transfer of blood containing toxins into solutions pumped into and removed from within the peritoneal organ cavity. A second form of dialysis treatment is a direct blood filtering process, whereby a needle or catheter is placed directly into a vein or artery, and through a series of connecting tubing, blood is removed, filtered and re-circulated back into the patient after filtration of the blood. These two hemodialysis procedures are the most common means for metabolic toxin removal from body fluids when a patient experiences total or bilateral renal failure.

Hemodialysis requires creation and maintenance of vascular access, which is the site on the body where blood will be removed and returned to a patient's body during dialysis. In some applications, such as dialysis, substantial volumes of fluid are circulated through the vascular system of a patient over a multi-year period. Without needle or indwelling catheter organ access for dialysis, there is no physical connection means to conduct dialysis toxin removal, and the ESRD patients would die within days of total renal failure. Hence, the insertion method and form of dialysis connection access relates directly to the patient's ability to have body fluids contained within an internal organ communicate and be safely connected “externally outside the body” for the purposes of blood hemofiltration.

Generally, some form of needle or indwelling catheter organ access is typically required as a physical connection means to conduct dialysis toxin removal. The insertion method employed and the form of dialysis access used affects the patient's ability to have body fluids contained within an internal organ communicate and be safely connected externally outside the body for the purpose of blood hemofiltration.

One approach to accessing the vascular system is the use of two catheters that are, at one end, each inserted in blood vessels and are routed out of the body through the skin, leaving six or more inches of catheter length outside the body for connection to a dialysis machine. However, the skin surrounding the holes where the catheters enter the body have trouble sealing around the catheters and can become infected. Another option for accessing the vascular system is creating an arteriorvenous (AV) fistula or graft for connecting an artery to a vein in the arm. However, the creation of an AV fistula or graft can be difficult or impossible in some patients.

In addition, because both types of dialysis treatment techniques discussed above require trained medical personnel for needle or catheter access and the administration of the actual external dialysis filtration process, there are significant health care hazards for both the patient and healthcare worker associated with such traditional needle access methods. Most ESRD patients must be transported to a public dialysis treatment center for treatment.

There are a number of additional complicating issues relating to the process of repeatedly sticking and cannulating a patient's circulatory organ system and removing/returning blood back to the patient. One significant complication is the need for maintenance of a sterile installation and connection technique for all components involved in establishing dialysis access through the patient's skin and into the hollow targeted organ, as well as connection to the dialysis tubing set, filter, and dialysis pump apparatus. Another complication is the inability of a particular artery or vein to be repeatedly cannulated or punctured at substantially the same convenient needle or catheter access site, due to vessel trauma, exit wound inflammation, dialysis graft complications, and/or enlarged needle hole formation resulting in massive needle hole bleeding/hematoma formation. Repeated dialysis needle punctures create unwanted scar tissue formation and infection. Both conditions are directly related to repetitive needle and catheter cannulation through the skin. Such large gauge needle cannulation complications are uncomfortable for the patient and the healthcare worker, due to the associated pain of additional needle sticks. Infection complications of these needle access sites are difficult to treat, because of the constant migratory effects of nosocomial infections, which have been well documented to routinely originate from direct contact with topical skin sourced Staphylococcocus bacteria. These frequently occurring needle access complications often require surgical intervention to repair, reconstruct, or remove the affected vessel organ, in addition to requiring extended hospital admission and costly medication treatment with IV medications.

One approach to the above problems is to provide a subcutaneous vascular access system that has a catheter connected to a blood vessel. The system is configured to receive a needle to provide access to the catheter. However, prior vascular access systems used for percutaneous needle cannulation for removal and return of blood during dialysis suffer many clinically devastating problems. Clinical problems include, but are not limited to, complications from poor tissue healing around the needle holes in the skin and subcutaneous needle tracks under the skin, caused by multiple puncturing of the same location, subcutaneous necrosis due to the proximity of the needle entrance to such devices, fluid contamination from leakage within the system and outside the needle entry, poor tissue coverage due to the shape and location of the indwelling permanent catheter connections as they exit the access system device, risk of infection caused by the compromised skin healing and leaking fluid during and after needle insertion, infection accumulation around the catheter connection and other complications.

One example of such an access system is a port vascular access assembly made by Vasca, Inc., described in U.S. Pat. No. 6,565,525. The Vasca, Inc., vascular access assembly requires two separate port units, one of which is used for input of blood back into the body and another for output of blood from the body. These ports are located just under the skin at a distance not far from each other, anchored to the subcutaneous tissue under the skin to inhibit movement, and require a sharp bevel tipped needle to be inserted in the same specific skin location and same perpendicular skin orientation every time. Use of the Vasca, Inc. vascular access assembly requires repeated punctures to skin, which is usually found to be stretched across the top of each port's needle entrance hole. Many clinicians who have experience with these devices have observed the patients' skin to be taut over the tops of these domed port housings, with minimal subcutaneous tissue found between the needle hole of the skin and the port opening which the needle enters the implanted port device. The condition of the skin with such devices has been observed to often include a poor blood supply condition of the skin because of the stretched condition of the skin, and the lack of adequate vascularized subcutaneous tissue to help fight infection and promote healing of the injured skin and subdermal tissue. Furthermore, the extremely short path of the needle through the skin to the opening of these port devices also provides less opportunity for the vascularized tissue to help control and prevent bacterial infection from occurring in and around the openings to the port opening. Moreover, because the needle punctures are occurring in the same skin location each time, infection and localized fluid accumulation abound the skin and device is common. The needle punctures create a clottable buttonhole over time, which is a skin injury that is not well controlled or completely sealed to the environment and therefore prone to repeatable, if not chronic infection. A further difficulty of conventional port access systems is their fluid filling chamber design, open bevel needle exposure to flowing blood fluids, causing massive blood cell damage and turbulent disruption to the blood by requiring sharp changes in fluid flow direction, such as a 90° change shortly just prior to fluid entering the exposed beveled edge of the needle, or just after exiting the open end of the needle as fluid enters the device fluid chamber.

### SUMMARY OF THE INVENTION

The present invention provides a subcutaneous needle connection system for providing access to a vascular system of a patient that has enhanced mechanical stability under the skin, improved sealing, improved fluid flow dynamics, improved needle hole healing potential of the skin and improved needle edge interfacing to minimize flow disturbance and blood cell trauma during use. The subcutaneous needle connection system is not a port chamber device like other early access systems, as it comprises a housing having a shaped bottom surface designed to facilitate stability of the subcutaneous needle connection system with a straight bidirectional fluid path. The subcutaneous needle connection system is connected to at least one catheter in communication with a vascular system of a patient and further includes one or more needle access openings for receiving a needle for accessing blood from the patient or returning treated blood to a patient for a procedure such as hemodialysis. The needle access openings may include a set of inner valves configured to automatically open upon insertion of a needle and a set of outer valves that may require manual activation in order to open, thereby ensuring enhanced sealing of the fluid paths.

The needle access openings may be angled away from each other to facilitate needle insertion and reduce operator error by preventing accidental insertion of the wrong needle in an opening. In addition, the surgically implanted housing may be manually rotated within the body or manipulated by an operator to increase the needle penetration zone are of the skin, reducing the need for repeated penetration of one area of skin during subsequent needle insertions into the needle connection system. Furthermore, the subcutaneous needle connection system may provide a substantially turbulent-free fluid flow path between one or both needle access openings and a corresponding catheter to maximize blood flow through the subcutaneous connection device.

According to one aspect of the invention, a subcutaneous needle connection system for providing access to a vascular system of a patient is provided. The subcutaneous needle connection system comprises a housing including a first needle opening configured to receive a needle, a second needle opening configured to be placed in communication with the vascular system and a first passageway connecting the first needle opening and the second needle opening. The system further includes a first valve disposed in the first needle opening, the first valve configured to automatically open upon insertion of a needle in the first needle opening to provide access to the first passageway. A second valve is also provided in the first needle opening that is configured to be mechanically activated to provide access to the first valve.

According to another aspect of the invention, a subcutaneous needle connection system comprises a housing, a first needle opening configured to receive a needle formed in the housing, a second needle opening configured to be placed in communication with the vascular system formed in the housing, and a first passageway extending through the housing and connecting the first needle opening and the second needle opening. The housing defines a bottom surface having three feet configured to rest on a subcutaneous surface within the patient.

According to still another aspect of the invention, a subcutaneous needle connection system for providing access to a vascular system of a patient comprises a housing and a needle mating system. The housing includes a first needle opening configured to receive a needle, a second needle opening configured to be placed in communication with the vascular system and a first passageway connecting the first needle opening and the second needle opening. The needle mating system is provided within the first needle opening for placing a needle inserted into the first needle opening in fluid communication with the first passageway, the needle mating system including a beveled surface configured to mate with a beveled surface on a needle inserted in the first needle opening.

### BRIEF DESCRIPTION OF THE DRAWINGS

The invention will be apparent from the description herein and the accompanying drawings, in which like reference characters refer to the same parts throughout the different views.

FIG. 1 illustrates an embodiment of a subcutaneous needle connection system implanted in a human body according to one aspect of the present invention;

FIG. 2 is a top view of the subcutaneous needle connection system of FIG. 1;

FIG. 3 is a bottom view of the subcutaneous needle connection system of FIG. 1;

FIG. 4 is a perspective view of the subcutaneous needle connection system of FIG. 1;

FIG. 6 is a side view of the subcutaneous needle connection system of FIG. 1;

FIG. 6 is a back side view of the subcutaneous needle connection system of FIG. 1;

FIG. 7 is a front side view of the subcutaneous needle connection system of FIG. 1;

FIG. 8 is a top cross-sectional view of the subcutaneous needle connection system of FIG. 1; and

FIG. 9 illustrates the inner and outer set of valves of a needle entry opening of the subcutaneous needle connection system of FIG. 1.

### DETAILED DESCRIPTION OF THE INVENTION

An illustrative embodiment of the present invention provides a subcutaneous needle connection system for providing access to a vascular system of a patient that exhibits enhanced mechanical stability under the skin, improved fluid flow dynamics, improved needle hole healing potential of the skin, improved needle edge interfacing to minimize flow turbulence and blood cell trauma during use, and other features. The invention will be described below relative to certain illustrative embodiments. Those skilled in the art will appreciate that the present invention may be implemented in a number of different applications and embodiments and is not specifically limited in its application to the particular embodiments depicted herein.

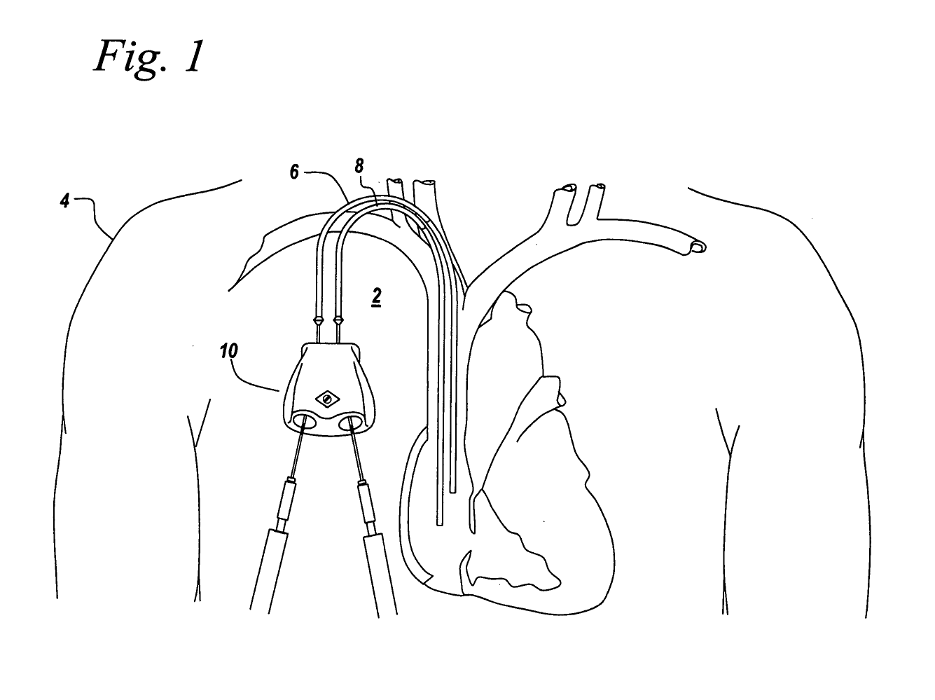

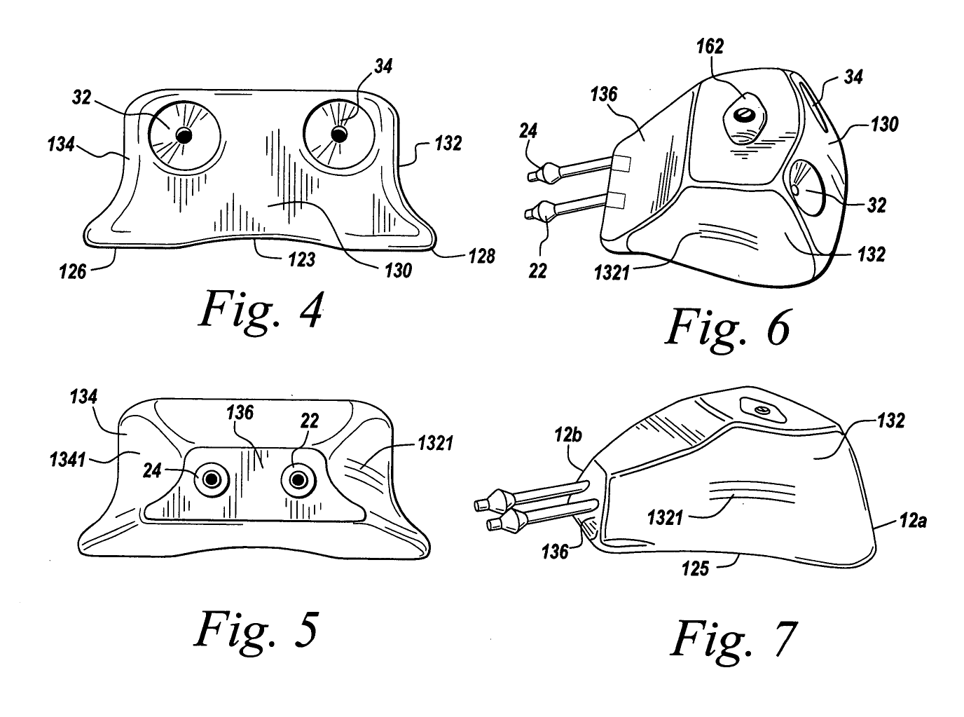

FIGS. 1-7 illustrates an embodiment of a subcutaneous needle connection system 10 for providing access to a vascular system of a patient according to an illustrative embodiment of the invention. The illustrative subcutaneous needle connection system ensures smooth, laminar flow of a body fluid, such as blood, accessed and/or returned to the body using the needle connection system, enhanced interfacing between needles, valves and fluid paths, leak-free needle connection capability, reduced operator error, minimal pain to a patient, minimal skin healing complications, and other improvements over vascular port access systems of the prior art.

As shown in FIG. 1, the illustrative needle connection system 10 may be configured to be implanted in a subcutaneous region of a patient, such as the chest 2 of a patient 4, as shown in FIG. 1. Preferably, the needle connection system 10 is completely embedded in the skin. When implanted in a patient, the needle connection system 10 is placed into communication with the vascular system via cannulas 6, 8 connected to one or more cannula openings of the needle connection system 10 and extending into a blood vessel 3 of the patient. The needle connection system 10 forms needle access openings, as described in detail below, for receiving needles that may be placed into communication with the cannulas via the needle connection system. A first needle inserted in the needle connection system may deliver treated fluid, such as blood treated by a dialyzer of a hemodialysis system, to the needle connection system. The first cannula 6 receives the treated fluid from the needle connection system and passes the treated fluid back to the vascular system. The second cannula 8 brings untreated fluid, such as blood from the vascular system, to the needle connection system 10. Another needle inserted in the needle connection system may receive and convey the untreated fluid provided by the second cannula 8 to the hemodialysis system or other treatment system.

According to an illustrative embodiment, the needle connection system may be used for receiving and returning treated blood during hemodialysis, though one skilled in the art will recognize that the needle connection system may have any suitable application requiring removal and return of a body fluid from and/or to a patient.

The illustrative subcutaneous needle connection system 10 preferably provides for parallel blood flow with a dual valve guided entry connection system. The needle connection system of the illustrative embodiment of the invention creates an ideally dynamic blood flow pattern, whereby uniform laminar flow and blood velocity remains continuous from the connection system entry to exit. Preferably, the blood follows a substantially straight path through the needle connection system 10, reducing disruptions caused by turns in the flow path.

The illustrative needle connection system 10 of the illustrative embodiment of the invention further facilitates the connection between the cannulas and the needles for accessing a body fluid. The needle connection system allows for improved tactile insertion of a percutaneous dialysis needle and for tactile confirmation of needle alignment. The illustrative needle connection system may include a plurality of sets of valves for controlling access to a needle access opening and preventing leaks. In one embodiment, the system may facilitate tactile needle alignment following tactile opening of a first mechanically operated valve.

The illustrative needle connection system may also provide a relatively large needle penetration area or “cannulation zone” so as avoid repeat needle entrance at the same location and to allow adequate repetitive dialysis needle cannulation site healing between each treatment. The relatively large penetration area provided by the illustrative needle connection system also provides a larger target for the needle, allowing some leeway during penetration.

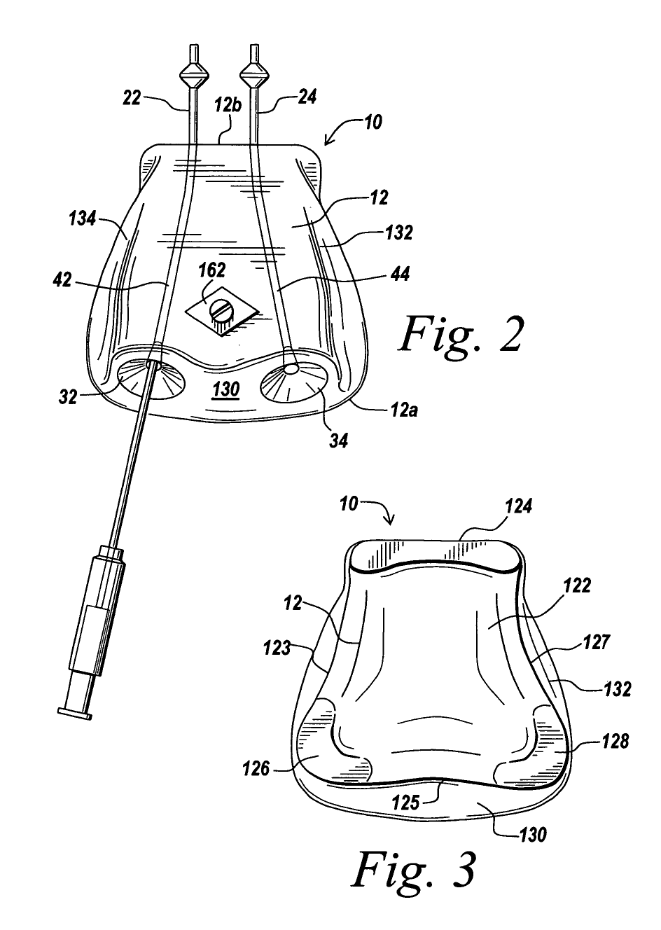

Referring to FIGS. 2-7, the illustrative subcutaneous needle connection system 10 comprises a housing 12 having a cannula end 12b for interfacing with the cannulas 6, 8 in communication with a vascular system. The illustrative cannula end 12b includes a first cannula opening 22 configured to be connected to a first cannula 6 connected to a blood vessel of a patient and a second cannula opening 24 to be connected to a second cannula 8 connected to a blood vessel of a patient. One of the cannula openings 22 may be an outlet for returning blood to the cannula 6 and vascular system from an associated needle inserted in the needle connection system, while the other cannula opening 24 may be an inlet for receiving blood from the vascular system and passing the received blood to an associated needle for treatment inserted in the needle connection system.

The housing 12 also includes a needle end 12a for interfacing with the needles inserted through the skin and connected to an outside hemodialysis system or other suitable system for treating the body fluid accessed by the needle connection system 10. The illustrative needle end 12a includes a first needle access opening 32 for receiving a first needle and a second needle access opening 34 for receiving a second needle. Passageways 42, 44 are formed within the housing 12 to selectively connect each needle access opening 32, 34, to an associated cannula opening 22, 24, respectively.

In the illustrative embodiment, one of the needle access openings 32 is associated via passageway 42 with the outlet cannula opening 22, and receives a needle for providing blood from a hemodialysis system to the needle connection system, which passes the treated blood to an associated cannula 6 for delivery to the vascular system. The other of the needle access openings 34 may receive a needle for receiving blood from an associated cannula 8 via the needle connection system 10 for delivery to the hemodialysis system for treatment, which then returns the treated blood to the body through the needle connection system 10. Preferably, the passageways 42, 44 provide for parallel blood flow entering and exiting the vascular system via the needle connection system 10. Valves are also provided within the passageways and/or needle access openings for selectively sealing the passageways and preventing fluid flow through the system after needle removal from the needle connection system, as described below.

The needle connection system 10 is preferably about the size of a pacemaker or smaller, i.e., less than about two inches across and less than about one-half an inch in thickness. The needle connection system 10 is installed surgically under the skin, in a location suitable for directed needle cannulation through the skin, and whereby a catheter can be inserted into the patient's vascular system and connected in a kink-free condition to the needle connection housing. The needle connection system housing preferably has a substantially pendulum-shaped configuration that tapers from the needle end 1 2a and the needle-connection openings to the cannula-connected end 1 2b and the cannula openings. When surgically installed in the body under the skin in a preferred needle accessible location, the needle connection system 10 is preferably located subcutaneously in an area which allows the implanted device to be manually rotatable or somewhat movable under the patients' skin by an operator to expose new skin areas for piercing by the needles for connection with the needle openings of the implanted housing, thereby increasing the needle cannulation zone of the skin. For example, one end of the needle connection system, such as the cannula end 12b may be stitched or otherwise fixed to the body, while the other end, such as the needle end 12a may be selectively movable by the operator to expose a new skin area prior to penetration of the needle into the tissue 12a. The increased needle penetration or cannulation zone allows a new skin area to be manually exposed and pierced by the operator to access the needle connection system, allowing for healing of each injured skin location without re-penetrating the exact same needle hole location. In this manner, the illustrative needle connection system may reduce or avoid repeated trauma to the same skin area each time the vascular system is accessed via the implanted needle connection system.

The needle connection system 10 of the illustrative embodiment of the invention creates an ideal dynamic blood flow pattern therethrough, whereby uniform laminar flow and blood velocity remains substantially continuous from the inlet openings 22, 32 to the outlet openings 24, 34. The openings may also be configured to facilitate alignment of a needle axis with a corresponding passageway axis to enhance blood flow through the needle access device. The alignment ensures uniform continuity of blood flow, blood flow at a constant speed as well as smooth physiologic flow. Preferably, the path has a substantially straight fluid path through the system to facilitate the smooth non-turbulent and bidirectional fluid flow. Taking the example of body fluid flow in a patient's blood vessels, each vessel runs generally parallel along the straight line direction of, e.g., the patients arm, leg, torso, or internal body cavity. It is desirable when body fluid is to be removed and/or re-circulated back into the patient's body for the body fluid within the needle access system to follow a generally straight line flow path that angles up and away from the surface of the cannula, then returns back into the patient at the same generally straight line flow path and divergent angle. As shown, the needle connection system provides a substantially straight fluid path between each cannula and needle, with all blood flow remaining substantially parallel to the direction of an aligned inserted needle. Thus, the needle connection system 10 directs the flow of blood away in a generally straight line flow path from the body fluid organs or blood vessels, and returns the blood to back to the blood vessels in the same generally straight line direction, without using sharp angles that could cause undue fluid turbulence and blood cell damage. Sharp, hard edge angles can adversely affect the natural flow dynamics of the blood, and damage fragile blood cell components, in addition to inducing chemical activation of certain blood containing components such as platelets, and circulating fibrinogen when blood component membranes are challenged by such turbulent forces.

The needle access openings 32, 34 for the needles are preferably formed in a side surface 130 forming the needle interface end 12a of the housing and the openings 22, 24 for the cannulas are preferably formed on a side surface 136 forming the cannula interface end 12b of the housing 12. The location of the openings, in particular the needle access openings 32, 34, on the side surfaces facilitates access to the openings, facilitating insertion of needles connected to a hemodialysis system into the needle connection system and also enables the substantially parallel, straight fluid flow paths. In addition, as described below, the provision of openings for interfacing with instruments outside of the body on the side surfaces of the system increases the cannulation zone and results in less stretching of the skin, thereby minimizing pain and infection.

The housing 12 may further include a topically recognizable manual actuator, illustrated as a manual valve activation button 162 formed on a top surface 124 thereof, as described in detail below, for selectively opening valves in the needle entry openings 32, 34 by the operator to allow insertion of a needle therein.

The housing 12 may be shaped to facilitate stability of the illustrative needle access system 10 when implanted in a patient. For example, the bottom surface 122 of the housing may be formed with a tripod-like shape to enhance stability. The illustrative tripod-shaped bottom surface 122 may be tapered from the needle interfacing end 12a of the housing toward the cannula interfacing end 12b, as described above, to form a substantially pendulum-shaped housing. As shown in FIG. 3, the tripod-shaped bottom surface 122 may include a first foot 124 located at the cannula end 12b of the housing, proximate the cannula openings 22, 24, and second and third feet 126, 128, respectively, located at the needle end 12a of the housing, proximate the needle access openings 32, 34. The second and third feet 126, 128 are disposed on opposite sides of the needle end 12a. The feet 124, 126, 128 may be in the form of a line, edge, protrusion or other suitable shape. The edges 123, 125, 127 connecting the feet 124, 126, 128, respectively, are preferably each formed in the shape of an arch, so that the bottom surface 122 may have a substantially concave shape.

The illustrative housing design provides enhanced stability relative to flat planar surfaces used in implants of the prior art. The design limits unintentional movement of the implanted needle connection system on the irregular surface on which it is placed, as movement of the system with the needles inserted therein during a transfer of blood would be dangerous. In addition, the tripod bottom surface 122 also withstands forces without compressing underlying tissue, thereby minimizing pain to the patient and minimizing obstructions to blood circulation. For example, the feet 124, 126, 128 lift much of the bottom surface up, off, and away from direct contact with the subcutaneous surface on which the device is placed. The illustrative shape also distributes downward loads to the center of housing 12, which further increases stability while helping to minimize subcutaneous tissue compression and pain to the patient during needle insertion. In contrast, the flat bottom surfaces used in implants of the prior art tend to be unstable on irregular surface, as well as prone to movement and compression of underlying tissue.

One skilled in the art will recognize that the bottom surface may alternatively have four feet, or another suitable shape configured to facilitate stability and is not limited to the illustrative tripod shape. For example, a subcutaneous needle connection system may have four or more feet on a bottom surface, a flat surface, a convex surface or any other suitable shape.

The side surfaces 132, 134 extending between the needle end 12a and the cannula end 12b of the housing 12 maybe configured with slight depressions 1321, 1341, respectively, to enhance the grip of a surgeon when stabilizing the needle connection system 10 to insert the needles into the needle access openings 32, 34.

The housing 12 may be formed of molded plastic, clear acrylic, titanium, or another suitable surgical material known in the art.

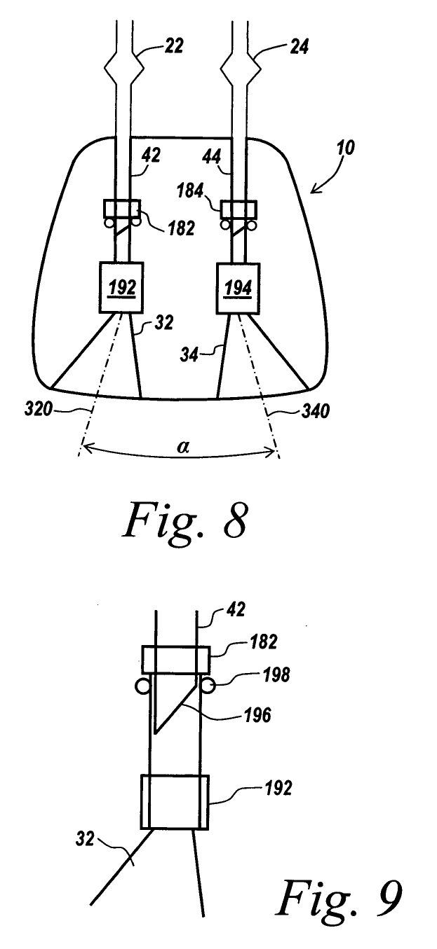

According to an illustrative embodiment of the invention, the illustrative needle connection system has a needle mating system within each needle access opening 32, 34 that includes an inner set of valves and an outer set of valves for controlling access to the needle access openings 32, 34 and associated passageways 42, 44, respectively. FIG. 8 illustrates the inner set of valves 182, 184 and outer set of valves 192, 194 respectively. FIG. 9 is a detailed view of one of the needle access openings 32 and associated passageways 42, having an inner valve 182 and an outer valve 192 for selectively sealing the opening and blocking, fluid flow therethrough.

The outer set of valves 192, 194 is preferably manually or mechanically operable by a user using a valve activator. In the illustrative embodiment, the outer valves 192, 194 require mechanical activation to open and automatically close in the absence of a mechanical activation to maintain the valves in an open position. For example, in an illustrative embodiment, the valve activator comprises the valve activation button 162 on the top surface of the housing, which may be depressed by a user to manually open the outer set of valves 192, 194 for the needle access openings 32, 34. The outer valves 192, 194 may be independently operated or a single operation may be used to operate both valves. According to an illustrative embodiment, each of the outer valves comprises a spring-loaded stopcock having a rotating cylinder that can not be opened unless the button 162 is depressed. After mechanical opening, the outer valves 192, 194 may be held open by a needle inserted in an associated opening 32 or 34 or by holding the button 162 in a depressed position. The outer valves 192, 194 preferably automatically close to maintain a leak-proof closure means when a user releases the valve activation button 162 and/or a needle is removed from the associated opening. One skilled in the art will recognize that any suitable means for manually or mechanically activating the outer valves may be used and that the invention is not limited to the illustrative valve activation button 162.

The inner set of valves 182, 184 may automatically open, for example, by inserting a needle into the respective opening after opening of the outer valves 192, 194 through mechanical means. Preferably, the inner set of valves does not automatically open with the opening of the outer set of valves, but rather, when a needle is inserted through the opened outer valves. In an illustrative embodiment, each inner valve comprises a sliding cylinder, opened by the needle tip itself. Upon removal of the needle, the inner valves automatically close and seal to form a leak-proof seal within the system 10.

The use of an outer set of valves that can only be manually opened and automatically close otherwise further prevents tissue from growing into the needle entrance when the dialysis needle is not engaged.

According to another aspect of the invention, each of the needle access openings 32, 34 is configured to facilitate a uniform, smooth connection with a corresponding passageway 42 or 44 for laminar flow of blood upon alignment of a needle with a respective opening. The needle access openings 32, 34 further provide for tactile feedback to the operator regarding the alignment of the needle. For example, as shown in FIG. 9, each needle access opening may include a beveled surface 196 configured to mate with a beveled surface of a needle designed to be inserted therein. The use of mating beveled surfaces provides improved tactile insertion of a percutaneous dialysis needle by providing tactile confirmation of the needle engagement and alignment to the operator. Preferably, the passageway 42 has an inside diameter that matches the diameter of a needle inserted in the associated needle access opening 32 to facilitate connection of the needle. The matching beveled edges when in alignment to each other, help to minimize blood cell damage when flowing over the leading edge of the engaged needle, further improving laminar flow through the system 10, with less trauma to the blood. In addition to providing tactile feedback that the needle has become fully engaged and aligned properly in the system, the matching beveled surfaces prevent twisting of the dialysis needle after the needle fully engages within the alignment area. The mating beveled surfaces further ensure complete alignment of the axis of the needle received in the needle access opening when the needle is gently pushed by the operator to its fully engaged and aligned stopping point.

The needle connection system may be configured to interface with an extended-length access needle. The extended length may enable insertion of the needle a substantial distance from the needle insertion openings, proving a substantially larger needle cannulation area of skin for spacing out needle holes. The tip of the needle is cut in a bevel best suited to mate with the bevel edge of the internal surface formed in the alignment area within the system, allowing an operator to feel the mating of the bevel edges when the needle is gently pushed forward to engage the beveled surfaces, by reducing rotational freedom about the axis of the needle. A needle inserted in the opening may have a slight bending in the tip to avoid scoring the inside of the valve. Preferably, when the needle is fully inserted in a needle access opening of the needle connection system, the needle cannot be rotated after the opposing beveled edges have become completely engaged

In one embodiment, the leading tip edge of a penetrating needle may serve to activate the automatically opening of an inner valve in the opening. One skilled in the art will recognize that any suitable means for automatically opening the inner valves may be used in accordance with the teachings of the invention.

The needle may be locked in place in the opening 32 through friction fit applied from the valves 182 and/or 192. In addition, a seal, such as an o-ring 198, may be disposed at the interface between the opening 32 and the passageway 42 to engage around the dialysis needle to provide a leak-proof connection.

The passageways 42, 44 may be generally tubular and can be further sized, dimensioned, or formed to either a conical or tapered shape, made generally smooth surfaced throughout, or made from several shorter faceted surfaces without sharp edges, and/or made coated, covered, or lined with medically purposeful bioactive substances (e.g. such as a coating or surface treatment that may contain one or more of an anticoagulant, antiseptic, gene therapy medication, anti-inflammatory medication, anti-proliferative medication, anti-biofilm or anti-microbial agent, a biological or lipophilic oil, or a hydrophilic fluid surface coating) to reduce needle insertion shear and friction resistance, or to reduce internal flow path fluid pressure resistance and flow rate resistance, internal flow path wall surface shear force, or to increase wall surface lubricity along all or a portion of passageways 42, 44, or to reduce the likelihood of circulating body fluid components and or blood cell components from being activated by direct surface contact with any portion of the passageway 42, 44 and including any portion of the needle access openings 32, 34 or cannula openings 22, 24. The coatings can be placed in all or partially in the areas exposed to body fluids. As described above, the passageways 42, 44 are preferably straight and aligned with the needle openings 32, 34 and cannula openings 22, 24 to promote smooth, laminar flow through the system 10.

According to another embodiment of the invention, as shown in FIG. 8, the needle access openings 32, 34 of the needle connection system 10 are divergent, i.e., angled away from each other, to facilitate insertion of needles therein. As shown, the axes 320, 340 of the openings 32, 34, respectively, are oriented at an angle a with respect to each other. The divergent angling of the openings away from each other and from each entrance site allows each needle entrance to be adequately located so as to avoid “operator error” by inadvertently inserting the wrong flow direction needle in the incorrect needle entrance. In addition, the divergent configuration of the openings 32, 34 increases the radius of the skin area available for cannulation, minimizing the injury to a needle site. One skilled in the art will recognize that the angle α between the axes of the needle access openings may have any suitable size, and that the invention is not limited to divergent openings.

Preferable, the needle openings are positioned in the middle or higher of the back side surface 130 to inhibit an inserted needle from traveling under the connection device 10.

According to another aspect of the invention, one or more of the external needle housing openings 32,34 can be substantially funnel-shaped to facilitate directional guidance of a needle tip into the center of the opening. The funnel shape increases the cannulation zone, and also provides a larger target area for guiding needles into the openings, requiring less precision when inserting a needle into each valve opening.

To use the needle connection system 10 of an illustrative embodiment of the invention, the needle connection system 10 is first implanted in a subcutaneous region in communication with the vascular system or other body fluid region. The system may be stitched to tissue to hold the system in a selected position, while allowing limited rotation or movement of the system in the subcutaneous region. For example, the cannula end 12b may be fixed, while allowing slight rotation of the needle end 12a relative to the cannula end 12b.

After implantation, needles are inserted into the needle connection system 10 to access the vascular system.

Prior to insertion of the needles, the system 10 may be angulated or rotated slightly, providing access to a wide area of skin that could be used to insert the needles, allowing for additional healing time before skin is punctured again. For example, the needle end 12a of the system 10 may angulate relative to the cannula end 12b, which may be fixed to the patient, in order to expose a new area of skin through which the needle can puncture in order to enter the valves.

In use, an operator will insert the first of two needles in the patient's skin. As the first needle approaches the associated needle access opening, the surgeon will depress the top button 162, opening the outer set of valves. As described above, each outer valve in the illustrative embodiment comprises a rotating cylinder that would not be opened unless the button is depressed. The insertion of the needle will automatically open the inner valve on the needle insertion side. The inner valve of the illustrative embodiment is formed by a sliding cylinder, opened by the needle tip itself. The operator continues to insert the needle through the second valve optionally rotating the needle slightly about its axis to detect the alignment mating of the beveled surfaces.

After insertion of the first needle, the second needle is inserted. The outer valve remains open because the first needle is holding it open, or the operator may optionally continue to depress the top button. The inner valve on the second side is opened by the tip of the second needle. Needle insertion continues until the bevels are mated. Laminar flow of blood or other fluids through the needle connection system is provided by a smooth transition from the needles to the associated catheter connection means.

As described above, because the needle access openings are located on a side of the device, the subcutaneous and dermal tissue punctured by the needle has adequate blood supply to help heal the injured tissue after needle withdrawal, as the tissue is not overly stretched and the device is not located next to or directly under the traumatized skin and subcutaneous tissue. The needle openings are also angled slightly outward, further separating the locations of the needle punctures from the skin, and the needle track locations of tissue through which the needle travels.

The illustrative needle connection system provides significant advantages over prior vascular access devices. For example, the needle connection system of the illustrative embodiment of the invention maintains uniform laminar flow of blood or another body fluid from entrance to exit. The system further provides improved tactile insertion of a percutaneous dialysis needle for tactile confirmation of complete needle alignment. The illustrative system further provides tactile entrance opening of the needle connection system with a rotating cylinder valve means and push button activator that automatically closes upon needle removal to maintain a leak-proof closure means. The illustrative connection system further provides for a permanent indwelling catheter attachment means to be directed downward, away from the skin, so as to prevent erosion of skin by the catheter connection means to the needle connection system. Another advantage of the invention is that the illustrative connection system provides for a large needle skin penetration area or “cannulation zone” to avoid repeat needle entrance at the same location, thereby allowing for healing between treatments. In addition, the illustrative needle connection system may reduce or prevent skin erosion due to contact with a catheter connection means by directing the permanent indwelling catheter attachment means downward, away from the skin. The illustrative needle connection system has an enhanced grip and feel, facilitating handling by a surgeon using the needle connection system to access the vascular system. The illustrative connection system also minimizes infection and pain while providing enhanced blood handling and other advantages not found or contemplated in vascular access devices of the prior art.

The present invention has been described by way of example, and modifications and variations of the exemplary embodiments will suggest themselves to skilled artisans in this field without departing from the spirit of the invention. Features and characteristics of the above-described embodiments may be used in combination. The preferred embodiments are merely illustrative and should not be considered restrictive in any way. The scope of the invention is to be measured by the appended claims, rather than the preceding description, and all variations and equivalents that fall within the range of the claims are intended to be embraced therein.

Having described the invention, what is claimed as new and protected by Letters Patent is:

1. A subcutaneous needle connection system for providing access to a vascular system of a patient, comprising:
a housing including a first opening configured to receive a needle, a second opening configured to be placed in communication with the vascular system and a first passageway connecting the first opening and the second opening;
a first valve disposed in the first opening, the first valve configured to automatically open upon insertion of a needle in the first opening to provide access to the first passageway; and
a second valve in the first opening configured to be mechanically activated to provide access to the first valve.

2. The subcutaneous needle connection system of claim 1, further comprising a topically recognizable manual actuator on the housing for selectively activating the second valve.

3. The subcutaneous needle connection system of claim 1, wherein the first valve automatically closes upon removal of a needle from the first opening.

4. The subcutaneous needle connection system of claim 1, wherein the first opening is formed on a side surface of the housing, the side surface extending substantially perpendicular to a surface on which the housing is placed when implanted in a patient.

5. The subcutaneous needle connection system of claim 1, wherein the housing has a bottom surface configured to interface with a surface of the patient when implants, the bottom surface having a tripod shape to facilitate stability of the system on the surface.

6. The subcutaneous needle connection system of claim 5, wherein the tripod shape includes a first foot in the vicinity of the second opening, a second foot in the vicinity of the first opening and a third foot in the vicinity of the first opening.

7. The subcutaneous needle connection system of claim 6, wherein the bottom surface include arcuate edges connecting the first, second and third feet.

8. The subcutaneous needle connection system of claim 1, wherein the first valve includes a beveled surface configured to mate with a beveled surface of a needle inserted in the first opening.

9. The subcutaneous needle connection system of claim 1, wherein the first opening is substantially funnel-shaped.

10. The subcutaneous needle connection system of claim 1, wherein the axis of a needle inserted in the first opening aligns with the first passageway and an axis of the second opening to provide a substantially straight fluid flow path through the housing.

11. The subcutaneous needle connection of claim 1, further comprising a third opening for receiving a needle, a fourth opening configured to be placed in communication with the vascular system and a second passageway connecting the third opening and the fourth opening.

12. The subcutaneous needle connection system of claim 11, wherein the first opening and the third opening are formed on a side surface of the housing and are angled away from each other.

13. The subcutaneous needle connection system of claim 1, wherein the housing is rotatable when implanted in the body to expose the first opening to different cannulation areas.

14. A subcutaneous needle connection system for providing access to a vascular system of a patient, comprising:
a housing;
a first opening configured to receive a needle formed in the housing;
a second opening configured to be placed in communication with the vascular system formed in the housing;
a first passageway extending through the housing and connecting the first opening and the second opening;
wherein the housing defines a bottom surface having at least three feet configured to rest on a subcutaneous surface within the patient.

15. The subcutaneous needle connection system of claim 14, wherein the first opening is formed on a first end of the housing and the second opening is formed on a second end of the housing opposite the first opening.

16. The subcutaneous needle connection system of claim 15, wherein a first foot is formed on the bottom surface in the vicinity of the second end of the housing and a second foot and a third foot are formed on the bottom surface in the vicinity of the first end of the housing.

17. The subcutaneous needle connection system of claim 15, wherein the bottom surface has a width that tapers from the first end to the second end.

18. The subcutaneous needle connection system of claim 14, wherein the bottom surface includes arcuate edges connecting each of the three feet.

19. The subcutaneous needle connection system of claim 14, wherein the first opening and the second opening are angled away from each other.

20. A subcutaneous needle connection system for providing access to a vascular system of a patient, comprising:
a housing including a first opening configured to receive a needle, a second opening configured to be placed in communication with the vascular system and a first passageway connecting the first opening and the second opening; and
a needle mating system within the first opening for placing a needle inserted into the first opening in fluid communication with the first passageway, the needle mating system including a beveled surface configured to mate with a beveled surface on a needle inserted in the first opening.

21. The subcutaneous needle connection system of claim 20, wherein the needle mating system includes a first valve configured to automatically open upon mating of the beveled surface of the needle with the beveled surface of the needle mating system.

22. The subcutaneous needle connection system of claim 21, wherein the first valve comprises a sliding cylinder valve.

23. The subcutaneous needle connection system of claim 21, wherein the needle mating system includes a second valve that is mechanically activated to provide access to the first valve.

24. The subcutaneous needle connection system of claim 23, wherein the second valve comprises a rotating cylinder valve.

25. The subcutaneous needle connection system of claim 21, wherein the first valve automatically closes when the needle is removed from the first opening.

26. The subcutaneous needle connection system of claim 20, wherein the first opening is substantially funnel-shaped.

27. The subcutaneous needle connection system of claim 20, wherein the needle mating system further comprises a sealing ring for engaging the needle upon mating of the beveled surface of the needle with the beveled surface of the needle mating system.

28. The subcutaneous needle connection system of claim 20, wherein the first opening is configured to align a needle with the first passageway and axis of the second opening upon mating of the beveled surface of the needle with the beveled surface of the needle mating system.
